(19) United States
(12) Patent Application Publication
(10) Pub. No.: US 2014/0205540 A1
(43) Pub. Date: Jul. 24, 2014

(54) Method of Improving Balance

(71) Applicant: Alan R. Hirsch, Riverwoods, IL (US)

(72) Inventor: Alan R. Hirsch, Riverwoods, IL (US)

(21) Appl. No.: 14/141,083

(22) Filed: Dec. 26, 2013

Related U.S. Application Data
(60) Provisional application No. 61/746,769, filed on Dec. 28, 2012.

(51) Int. Cl. A61K 35/00; A61K 49/00

(52) U.S. Cl. CPC A61K 35/00; A61K 49/0004
USPC 424/9.2; 514/789

(57) ABSTRACT

Methods and compositions for modifying an individual's balance time are disclosed.

### FIELD OF THE INVENTION

Embodiments of the invention relate to methods of improving the balance of an individual.

### BACKGROUND OF THE INVENTION

Dizziness or instability affects almost 70 million Americans, and disorientation caused by the loss of balance and/or dizziness can pose a great danger to an individual. It also presents an additional obstacle to many individuals undergoing physical rehabilitation and therapy.

The ability to maintain balance is important in daily life, and it would be useful to provide a means of improving an individual's balance that is non-invasive, convenient, safe, and easy to administer.

### SUMMARY

In one aspect, the invention provides methods of improving the balance of an individual.

In one embodiment, the method comprises administering to the individual for inhalation a composition comprising an odorant, wherein the inhalation of the odorant composition is effective to increase the amount of time the individual can maintain their balance while performing a balance activity compared to the amount of time they can maintain their balance while performing the same activity but without having inhaled the odorant composition.

In another embodiment, the method comprises administering to the individual for inhalation a composition comprising an odorant, having the individual perform a balance activity with and without inhaling the odorant composition, and recording and comparing time amounts (“balance time”) during which the individual maintains balance during the balance activity with and without inhaling the composition, wherein the inhalation of the odorant composition is effective to increase the time amount (balance time) by a statistically significant amount.

In embodiments, the individual can perform the balance activity with and without inhalation of the odorant composition repeatedly two or more times, and an average of the time amounts (with and without inhaling the composition) can be compared to determine the statistical significance of the balance time associated with inhaling the composition.

In embodiments, the composition comprises a concentration of the odorant greater than an average normal threshold concentration of the odorant. In embodiments, the composition comprises a suprathreshold but non-irritant concentration of the odorant. In embodiments, the composition comprises a concentration of the odorant of about 25-55 decismel units.

In preferred embodiments, the composition comprises a cinnamon buns odorant in an amount or concentration effective to increase the time amount (balance time) of the individual in performing a balance activity. In another embodiment, the composition comprises an effective amount of a cinnamon odorant and a vanilla odorant to increase the time amount (balance time).

In embodiments, the composition consists essentially of a cinnamon buns (sweet cinnamon vanilla) odorant in a carrier, and optional additives. In other embodiments, the composition comprises a mixture of odorants in a carrier, the odorant mixture comprising a cinnamon buns odorant as the primary odorant in combination with one or more secondary odorants that complement and do not mask the primary odorant, such that, when inhaled by an individual, the composition is effective to increase the time amount (balance time) by a statistically significant amount. In embodiments, the cinnamon buns (primary) odorant is present at a suprathreshold and non-irritant concentration and the secondary odorant(s), if present, is at a less than suprathreshold concentration.

In another embodiment, the composition consists essentially of a mixture of a cinnamon odorant and a vanilla odorant in a carrier, and optional additives. In another embodiment, the composition comprises a mixture of odorants in a carrier, the odorant mixture comprising a mixture of a cinnamon odorant and a vanilla odorant as the primary odorant, in combination with one or more secondary odorants that complement and do not mask the cinnamon odorant/vanilla odorant mixture (primary odorant), such that, when inhaled by an individual, the composition is effective to increase the time amount (balance time) by a statistically significant amount. In embodiments, the mixture of the cinnamon odorant and vanilla odorant (primary odorant) is present at a suprathreshold and non-irritant concentration and the secondary odorant(s), if present, is at a less than suprathreshold concentration.

In embodiments, the method further includes assessing the time amounts (balance times) of the performance of the balance activity with and without inhaling the odorant composition to determine effectiveness of administering the odorant composition to improve the individual's balance.

In embodiments, the method can be self-administered. In other embodiments, the method can be administered by a third person.

In another embodiment, a method of improving balance of an individual according to the invention, comprises inhaling a composition comprising an odorant and performing a balance activity, wherein inhaling the odorant composition is effective to increase the time amount (balance time) during which the individual maintains balance performing the balance activity by a statistically significant amount compared to a time amount (balance time) during which balance is maintained performing the same balance activity under the same conditions but without inhaling the odorant composition.

In another embodiment, a method of improving balance of an individual according to the invention, comprises the steps of: (A) Having the individual perform a balance activity; (B) Recording a first time amount during which the individual maintains balance during the activity; (C) Administering to the individual for inhalation a composition comprising an odorant or mixture of odorants; (D) Having the individual perform the balance activity; (E) Recording a second time amount during which the individual maintains balance during the balance activity; and (F) Scoring the difference between the first and second time amounts; wherein the inhalation of the odorant composition is effective to increase the second time amount by a statistically significant amount compared to the first time amount.

In another embodiment, steps (C) and (D) are performed prior to steps (A) and (B). For example, the method can comprise the steps of (A) Administering to the individual for inhalation a composition comprising an odorant or mixture of odorants; (B) Having the individual perform a balance activity; (C) Recording a first time amount during which the individual maintains balance during the balance activity; (D) Having the individual repeat the balance activity without inhalation of the composition; (E) Recording a second time amount during which the individual maintains balance during the repeat balance activity; and (F) Scoring the difference between the first and second time amounts; wherein the inhalation of the odorant composition is effective to increase the time amount by a statistically significant amount.

In another aspect, the invention provides an article of manufacture (or kit). In an embodiment, the article (kit) includes, packaged together, a container of a composition comprising an odorant, and instructions for administration of the composition to an individual by inhalation for improving the individual's balance. In a preferred embodiment, the composition comprises a cinnamon buns odorant as the primary odorant. In an embodiment, the composition consists essentially of the cinnamon buns odorant. In another embodiment, the composition comprises a mixture of the cinnamon buns odorant as the primary odorant, with a secondary odorant, i.e., an odorant that enhances but does not mask the primary odorant, or is hedonically neutral to the subject individual.

In another embodiment, the article of manufacture (kit) comprises a container of a composition comprising a mixture of a cinnamon odorant and a vanilla odorant as the primary odorant. In an embodiment, the composition consists essentially of the cinnamon odorant/vanilla odorant mixture. In another embodiment, the composition comprises a mixture of the cinnamon odorant/vanilla odorant mixture as the primary odorant, with a secondary odorant.

The article (or kit) can further include a device for administering a plurality of odorants for testing olfactory ability of the individual, a device for administering a series of odorants for testing olfactory threshold of the individual, a set of odorants for testing an individual's olfactory ability, and/or a set of serial dilutions of an odorant for testing olfactory threshold.

In another aspect, the invention provides methods of screening a test odorant composition for effectively increasing an individual's balance time. In one embodiment, a screening method includes having an individual inhale a test odorant composition consisting essentially of the odorant or odorants to be tested; instructing the individual to perform a balance activity with and without inhaling the test odorant composition; recording and comparing time amounts during which the individual maintains balance during the balance activity with and without inhaling the composition; determining statistical significance between the two time amounts; and eliminating the test odorant composition as being ineffective if the time amount of performing the balance activity with inhaling the composition is not increased by a statistically significant amount compared to the time amount without inhaling the composition. In embodiments, the screening method includes having the individual inhale an inactive odorant as a control odorant composition.

In another embodiment, a method of screening a test odorant composition for improving an individual's balance, the test odorant composition consisting essentially of the odorant or odorants to be tested, includes the steps of: having the individual perform a balance activity without inhalation of the test odorant composition and measuring the interval of time during which the individual maintains balance as a “control” time; having the individual perform the same balance activity under the same conditions with inhalation of the test odorant composition and measuring the interval of time during which the individual maintains balance as a “test” time; comparing the control time to the test time to determine the statistical significance of the test time; assessing the composition as effective to improve the balance time of the individual based on the statistical significance of the test time; and eliminating the odorant composition as ineffective to improve the individual's balance if the test time is not statistically significant (p<0.05). In embodiments, the step of having the individual perform the balance activity without inhalation of the test odorant composition is performed after the step of having the individual perform the balance activity with inhalation of the test odorant composition. In other embodiments, the steps of having the individual perform the balance activity with and without inhalation of the test odorant composition are repeated at least two times, and an average of the test time amounts and an average of the control time amounts are compared to determine the statistical significance of the test time.

### DESCRIPTION OF THE INVENTION

The present invention relates to methods of using odorants to improve an individual's ability to maintain balance and/or performing a skill or activity requiring balance. The methods involve administering a composition comprising an odorant or mixture of odorants for inhalation by the individual into his/her nasal passageway. In embodiments, the method involves delivering an odorant composition composed of an effective amount or concentration of an odorant or mixture of odorants to an individual for inhaling to improve or enhance their ability to maintain balance compared to their ability to do so without having inhaled the odorant composition. The method can be utilized with both male and female individuals.

The term “balance time” as used herein refers to a period of time that an individual maintains balance during performance of a balance activity.

The method can be utilized to increase the time period of maintaining balance (balance time) by an individual during an activity requiring balance (i.e., a balance activity), for example, to facilitate physical therapy and/or rehabilitation, or to enhance, for example, athletic or job performance. For example, a composition composed of a hedonically favorable odorant (e.g., cinnamon buns odorant) could be used in a therapy for those who suffer from dizziness and/or instability. Clinical applications can include administering the composition, for example, to patients undergoing physical therapy and/or rehabilitation to quell dizziness and/or improve their balance.

In embodiments of the invention, a composition composed of an effective amount of an odorant or mixture of odorants can be administered for inhalation to the individual prior to or during performance of a balance activity to cause an increase in the length of time (“time amount”) for which the individual maintains their balance during performance of the balance activity.

Non-limiting examples of a balance activity include standing erect on both feet, standing erect on both feet and raising one leg forward or to the side, standing on a balance board, repeatedly sitting and then standing up, walking back and forth across a room, walking up and down a slope or stairs, standing with feet shoulder-width apart and repeatedly leaning forward and then backward shifting the body weight to the toes and then the heels, among other activities. A balance activity can be performed with eyes open or eyes closed.

In embodiments, the composition can comprise one or more odorants that the subject individual considers to be hedonically pleasant (positive). In other embodiments, the composition can comprise one or more odorants that the subject individual considers to be hedonically pleasant combined with one or more odorants that the subject individual considers to be hedonically neutral.

In embodiments of the invention, a composition that is considered to be hedonically pleasant to an individual and which comprises a hedonically pleasant odorant as the primary or dominant odor (aroma) of the composition, solely or in a mixture of odorants, can be administered to the individual to inhale, to increase the individual's balance time compared to the individual's balance time without inhaling the hedonically pleasant composition, by a statistically significant amount (p<0.05). In some embodiments, such a mixture of odorants is composed of the primary or dominant odorant in combination with one or more secondary odorants that enhance and/or complement and do not mask the primary odorant or are considered to be hedonically neutral to the subject individual.

In preferred embodiments, the composition comprises an effective amount of a cinnamon buns (sweet cinnamon vanilla) odorant as the primary or dominant odor (aroma), alone or in combination with one or more secondary odorants. In some embodiments, the composition consists essentially of an effective amount of a cinnamon buns odorant.

In other embodiments, the composition comprises a mixture of effective amounts of a cinnamon odorant and a vanilla odorant as the primary or dominant odor (aroma) (i.e., as a sweet cinnamon vanilla odor), alone or in combination with one or more secondary odorants. In some embodiments, the composition consists essentially of an effective amount of a mixture of a cinnamon odorant and a vanilla odorant.

The composition can be inhaled by the subject individual prior to or during performing a balance activity to result in an increase in the time period (duration) during which the individual maintains their balance compared to the time duration without having inhaled the composition. In some embodiments, the composition is inhaled by the individual prior to and during the balance activity.

As used herein, the term “odorant” refers to an odor-causing chemical compound or mixture of compounds that, when delivered in a gaseous or aerosol medium, can stimulate olfactory and/or trigeminal chemoreceptors in the nasal cavity and cause a physiological or psychological response. A hedonically pleasant or positive composition, odorant or odorant mixture is one to which the subject individual has a pleasant or positive reaction to its scent. A hedonically negative composition, odorant or odorant mixture is one to which the individual has a repulsive or negative reaction to its scent. A hedonically neutral composition, odorant or odorant mixture is one to which the individual has neither a positive nor negative reaction.

In a preferred embodiment, the subject individual is presented with the composition containing a suprathreshold concentration (e.g., about 25-55 decismel units) of the odorant or odorant mixture that is near but not so high as to become an irritant (trigeminal), which the individual inhales prior to exposure to a sensory stimulus. The level or concentration of the odorant or odorant mixture within the composition and/or mode of administering the composition is sufficient to overcome competing or conflicting ambient odors that may act to nullify its effect.

An odorant is presented at a “suprathreshold” level when the decismel level or concentration of the odorant is beyond that needed to be detected by a normosmic individual. At its irritative level, the odorant quantity is so high and intense that the odorant stimulates predominantly the trigeminal nerve (for pain) rather than the olfactory nerve and, hence, is perceived as noxious or painful. The irritation threshold of the patient is the lowest concentration of the substance that causes immediate stinging or burning sensations in the nose, or stinging or lacrimation of the eye. (See, J. F. Gent, in Clinical Measurement of Taste and Smell, pages 107-166, H. L. Meiselman et al. (eds.), 602 pp., MacMillan, NY (1986); R. L. Doty et al., Ann. Neurol. 25: 166-171 (1989); E. Koss et al., Neurology 38: 1228-1232 (1988); and R. Doty, The Smell Identification Test: Administration Manual 1983: 13-14, Philadelphia: Sensonics, Inc. (1983)).

If desired, prior to the administration of the odorant, the subject individual can undergo olfactory testing according to a test such as the University of Pennsylvania Smell Identification Test (UPSIT), a 40-question forced-choice, scratch-and-sniff identification test (R. Doty, The Smell Identification Test: Administration Manual 1983: 13-14, Philadelphia: Sensonics, Inc. (1983)), the Chicago Smell Test, a 3-item detection and identification test (A. R. Hirsch et al., Chemical Senses 18(5): 570-571 (1993); A. R. Hirsch et al., Chemical Senses 17(5): 643 (1992)), the Quick Smell Identification Test (Q-SIT), a three (3) question scratch-and-sniff smell identification test (Sensonics, Inc., Haddon Heights, N.J.), or other detection and/or identification test.

The subject individual can also be evaluated for olfactory capacity (e.g. loss of smell) according to an olfactory threshold test as known and used in the art. Such a test provides a precise magnitude of loss of smell and classifies the individual as normosmic, hyposmic or anosmic, which is useful in assessing the effectiveness of a particular odorant and/or the required concentration of the odorant, preferably a suprathreshold and near but below irritant level, to provide the desired effect according to the method of the invention. In the embodiment of that test, an odorant substance such as butyl alcohol, phenyl ethyl alcohol, or pyridine, is combined in an odorless liquid medium to provide a series of dilutions, or binary steps, of the odorant. For each successive binary step up the dilution scale, the odorant is present, for example, at one half the concentration of the preceding step. The highest concentration of the odorant usually provides the substance at an irritant level. The individual is presented with the series of dilutions in ascending order, and is asked to compare each dilution step to at least one control stimulus, such as odorless propylene glycol.

In the art, a “normosmic” individual is one who can detect the odor of a substance without irritant sensations when the odorant is presented with the range of its average normal threshold. A “hyposmic” or “microsmic” individual has reduced capacity of the olfactory nerve being able to detect an odorant substance by its odor at a concentration, or decismel level, above that of a normosmic individual yet below its irritant concentration level. An “anosmic” individual is one who has essentially no olfactory nerve capacity being unable to detect the odor of the odorant substance, but has trigeminal nerve function, being able to detect an odorant substance by means of irritant, tingling sensations when it is present at an irritant concentration. A patient who is able to detect pyridine vapor by means of irritant, tingling sensations caused by stimulation of the trigeminal nerve, but who cannot distinguish a pyridine odor at a lower concentration without such sensation, is considered to be anosmic having no olfactory nerve sensitivity.

Ranges of the average normal threshold for various odorant substances can be found in the art, for example, Amoore and O'Neill, “Proposal for Unifying Scale to Express Olfactory Thresholds and Odor Levels: The “Decismel Scale”,” in Proceedings of the 1988 Air Pollution control Association Annual Meeting, Paper No. 78.5 (21 pp.), Air and Waste Management Association, Pittsburgh, Pa. (1988); Amoore and Haotala, “Odor as an Aid to Chemical Safety: Odor Thresholds Compared with Threshold Limit Values and Volatiles for 214 Industrial Chemicals in Air and Water Dilution,” J. Appl. Toxicology 3(6):272-290 (1983).

A suprathreshold amount is a concentration of the odorant/odorant mixture that is greater than the average normal threshold concentration of the odorant or mixture. The normal threshold concentration can be determined by administering a series of the same concentrations of the odorant/odorant mixture to a control group of at least 25 individuals who do not have a chemosensory dysfunction, and calculating the mean threshold concentration detected by the group of 25 individuals. Another alternative is to refer to the known threshold concentration value for the odorant/odorant mixture that has been established previously and published by J. Amoore et al., J. Appl. Toxicology, 3:272 (1983).

Odor thresholds can be expressed on the decismel scale. The decismel scale is constructed by setting the mean threshold concentration of a chemosensory agent detected by the control group of 20 year olds at the “0” value. A decismel is calculated by dividing the concentration of the odorant detected by the patient by the normal threshold concentration (using the published value or empirically determining the value) and then taking the logarithm of the quotient. The logarithm of the quotient is then multiplied by 20 to obtain the decismel value. Decismel values can be positive or negative. A positive decismel value indicates the patient is less sensitive to the odorant, i.e. has a higher threshold detection concentration. A negative decismel value indicates that the patient is more sensitive to the compound, i.e. has a lower threshold detection concentration. An increase in the threshold concentration value over the mean threshold concentration value of 2 fold corresponds to 6 decismels (or ds). Determination of decismel units is known in the art, as addressed, for example, in U.S. Pat. Nos. 5,380,765 and 5,492,934 (Hirsch).

The odorant compositions used in the methods of the invention are considered to be hedonically positive by a subject individual and contain an effective amount of an odorant or mixture of odorants such that, when inhaled by the individual, the amount of time or time interval (balance time) that the individual maintains balance during a balance activity is substantially enhanced (increased) compared to the balance time of performing the same balance activity under the same conditions but without having inhaled the odorant composition. Such an effect can be objectively assessed by measuring and comparing the individual's balance time for a balance activity both with and without the effect or inhalation of the odorant composition. The balance time can be measured from a start time of performing a balance activity to an end time when the individual is unable to maintain their balance, i.e., has a loss of balance.

The concentration of the odorant or mixture of odorants is preferably at a suprathreshold concentration and preferably near but not an irritant concentration at a decismel level of about 25-55 decismel units, preferably greater than 25 decismel units, preferably at about 30-55 decismel units.

In embodiments of the invention, the hedonically pleasant/positive odorant composition contains an effective amount of a cinnamon buns (sweet cinnamon vanilla) odorant as the dominant (primary) odor or essence such that, when inhaled, the subject individual's balance time in performing a balance activity is increased compared to the individual's balance time performing the same balance activity but without having inhaled the odorant composition. In another embodiment, the composition contains an effective amount of a mixture of a cinnamon odorant and a vanilla odorant as the dominant odor or essence.

A preferred odorant composition for use in increasing an individual's balance time is a formulation in which the dominant odorant component is a cinnamon buns odorant and eliminates odorants and other components that compete with the cinnamon buns odorant accords or notes to provide a full effect on the individual inhaling the odorant composition. In some embodiments, the composition consists essentially of the cinnamon buns odorant. In other embodiments, the cinnamon buns odorant can be combined with one or more secondary odorants that enhance but do not mask the cinnamon buns odorant, or are hedonically neutral to the subject individual.

In another embodiment, the odorant composition is a formulation in which the dominant odorant component is a mixture of a cinnamon odorant and a vanilla odorant, and eliminates competing odorants and other components that compete with the accords or notes of the cinnamon/vanilla odorant mixture. In some embodiments, the composition consists essentially of a mixture of a cinnamon odorant and a vanilla odorant. In other embodiments, the cinnamon/vanilla odorant mixture can be combined with one or more secondary odorants that enhance but do not mask the cinnamon/vanilla odorant mixture, or are hedonically neutral to the subject individual.

In embodiments of the invention, the odorant or odorant mixture is provided as a formulated composition of a single essential (primary or dominant) odorant or, in other embodiments as a blend (mixture) of two or more essential odorants, to cause the desired effect, and eliminates odorants and other components that compete with or mask the effective odorant(s). In some embodiments, the composition is composed of a blend of one or more essential (primary) odorants with one or more secondary odorants that do not compete with or mask the essential odorant(s), and/or are hedonically neutral to the subject individual, and in some embodiments enhance the essential odorant(s). The odorant composition can be formulated by combining the essential odorant(s) alone or as a blend with a secondary odorant(s) with an odorless carrier such as mineral oil or water, and odorless additives such as preservatives and the like. The odorant composition can be formulated with a viscosity effective to allow for aerosolization or to provide a thick liquid material, e.g. a gel or cream.

In one embodiment, the composition consists essentially of a suprathreshold and non-irritant concentration of one or more odorants such that, when inhaled by an individual, the composition is effective to improve (e.g., increase) the balance time of the individual in the performance of a balance activity by a statistically significant amount compared to the individual's balance time in performing the same balance activity but without inhalation of the composition. For example, the composition can consist essentially of a cinnamon buns odorant in a carrier with optional additives such that, when inhaled by an individual, the composition is effective to increase the individual's balance time for a balance activity by a statistically significant amount compared to the individual's balance time performing the same balance activity but without inhaling the composition. In another embodiment, the composition can consist essentially of a mixture of cinnamon and vanilla odorants in a carrier with optional additives.

In other embodiments, the composition can be composed of a mixture of odorants in a carrier with optional additives, for example, a suprathreshold and non-irritant concentration of a cinnamon buns odorant as the primary odorant, in combination with a less than suprathreshold concentration of one or more secondary odorants, such that, when inhaled by an individual, the composition is effective to increase the individual's balance time in the performance of a balance activity by a statistically significant amount compared to the individual's balance time performing the same balance activity without having inhaled the composition. In another embodiment, the composition can be composed of a suprathreshold and non-irritant concentration of a mixture of cinnamon and vanilla odorants in a carrier with optional additives, and a less than suprathreshold concentration of one or more secondary odorants.

Odorants for use in the present methods are commercially available as a liquid, essential oil, extract, or other form from a variety of sources. Nonlimiting examples of such sources include, for example, International Flavors & Fragrances Inc. (IFF) (New York, N.Y.), and Creative Fragrances, LTD (Dallas, Tex.), among others.

The odorant composition is preferably formulated as a liquid solution or a spray, but can also be provided in the form of a cream, lotion, or other consistency, and can be contained within a liquid pump device, aerosol or non-aerosol spray device, lidded container, a blister pack, or other suitable vessel such as those known and used in the art. The odorant composition can also be contained in a solid form within a capped vessel. It is preferred that the odorant composition is provided in a portable dispenser that is easily transportable and readily accessible.

In embodiments of conducting the method of the invention, the odorant composition can be administered for inhalation by the subject individual prior to performing a balance activity to increase the individual's balance time in performing the activity. For example, to increase the balance time, an odorant composition can be administered to an individual for inhalation and the individual can then perform one or more balance activities. In some embodiments, the method can involve two or more sessions during which the individual inhales the odorant composition and then performs one or more balance activities, and measuring and assessing the individual's balance time over the two or more sessions. A comparison can also be made with the balance time of the individual performing the balance activity but without inhalation of the odorant composition (i.e., as a control). The time period, for example, from the start of the balance activity to the time at which the individual's balance is lost, can be measured to provide the balance time, which can be compared to a control and/or other time interval(s) measured for the same balance activity to assess improvement in balance time.

Administration of the odorant composition can be achieved by bringing an effective amount and concentration of the odorant composition into proximity of the individual for inhalation, for example, by spraying, by applying the odorant composition to a piece of clothing of the individual or directly to the face of the individual below the nostrils, or to a cloth or paper material such as a mask (e.g., a surgical mask, dust-type mask, earloop face mask, and the like) that is then secured over the nostrils of the subject individual, among other techniques. In another embodiment, the composition can be administered by means of a flexible laminate material (e.g., patch) sized to fit beneath the nose that incorporates the odorant composition and has a pressure-sensitive adhesive layer (covered by a release layer) that allows the material to adhere to skin and which is positioned under the nostrils of the individual, as described, for example in U.S. Pat. No. 6,769,428 (Cronk). In yet another embodiment, the odorant composition can be administered through the use of a portable delivery device operable to provide continuous delivery of a vaporous emission of the odorant composition through cannulla (tubes) inserted into the nostrils of the individual as described, for example, in U.S. Pat. No. 6,803,987 (Manne). Other delivery systems can be used for delivery of the odorant composition to the individual.

Odorants or odorant mixtures can be readily screened and assessed for effectiveness in improving an individual's balance according to the invention. For example, a composition containing an odorant or mixture of odorants can be administered to an individual for inhalation to evaluate its effect on increasing the individual's balance time in performing a balance activity by comparing the balance time to the individual's balance time in performing the same balance activity but without inhaling the odorant composition. In some embodiments, the individual can inhale an inactive odorant or neutral odorant as a control odorant composition. Elmes et al., Adequacy of Control Comparisons in Olfactory Experiments. Chem. Percept. 1:247-252 (2008). For example, an individual can be instructed to perform a balance activity and the interval of time from the start of the activity to the individual's loss of balance (without inhaling the test odorant composition) can be measured as a “control.” The subject individual can then inhale a test odorant composition, the balance activity can again be performed and the time interval from the start of the activity to the individual's loss of balance can be measured and compared to the control balance time interval. Optionally, the individual can also be questioned as to a positive or negative reaction to the pleasantness of the scent to assess the hedonics of the odorant composition.

Another embodiment of a method of screening a composition formulated with an odorant or a mixture of odorants for effectively enhancing an individual's balance time can comprise the steps of:
a) Having an individual perform a balance activity without inhalation of the test odorant composition and measuring the time interval from the initiation of the activity to the subject individual's loss of balance as a “control” time;
b) Having the individual inhale a composition consisting essentially of the odorant or odorants to be tested, and then re-perform the same activity, and then measure the interval of time from the initiation of the activity to the subject individual's loss of balance to provide a “test” balance time;
c) Comparing the control balance time to the test balance time to determine the statistical significance between the two balance times; and
e) Eliminating the odorant or odorant mixture as ineffective to enhance the individual's balance time if the test balance time is not statistically significant (p<0.05).

In embodiments of a screening method, step b) can be performed before step a). The screening test as well as the method of the invention can include other steps such as having the inhaling individual identify the composition as hedonically positive, neutral or negative, and testing olfactory ability and/or olfactory capacity of the individual, among other olfactory tests known and used in the art.

According to the invention, a composition comprising the odorant or odorant mixture is administered (e.g., dispensed) as a vaporous emission to the nostrils of an individual for inhalation of a concentration of a primary odorant or odorant mixture effective to improve the individual's balance, for example, using a cinnamon buns odorant-based composition. As another example, the composition is composed of a mixture of a cinnamon odorant and a vanilla odorant. Such an effect can be assessed and measured objectively by comparing the individual's balance time in performing a balance activity with and without the administration of the odorant composition.

The odorant composition can be packaged as part of an article of manufacture, or kit. In one embodiment, the article of manufacture can comprise a container of an odorant composition or, packaged together, a container of a first odorant and a container of a second odorant (etc.) for combining together to form the odorant composition. The odorant composition comprises an odorant or mixture of odorants in a suprathreshold and but non-irritant concentration, and preferably near a non-irritant concentration, effective to substantially increase an individual's balance time when administered according to the method of the invention and performing a balance activity.

In a preferred embodiment, the composition consists essentially of a cinnamon buns odorant. In another embodiment, the composition consists essentially of a cinnamon odorant and a vanilla odorant as a mixture. For example, the article of manufacture can comprise a container of an odorant composition consisting essentially of a cinnamon buns odorant. In another example, the article can comprise a container of one or more odorants of which a cinnamon buns odorant is the dominant (primary) odor or essence. As another example, the article of manufacture can comprise a container of an odorant composition consisting essentially of a mixture of a cinnamon odorant and a vanilla odorant as the dominant (primary) odor or essence, optionally with one or more secondary odorants. The article of manufacture can further include a device for use in delivery of the composition to a subject individual, for example, a mask for placement over the nose of the individual, a device for applying the composition directly to the skin under the nostrils of the individual, a spray delivery device, among others.

The article of manufacture(kit) can further include one or more elements for testing the individual, that can be separately packaged, including a device for administering odorant(s) for testing olfactory ability of the individual (e.g., UPSIT), and/or a device for administering a series of odorants for testing olfactory threshold of the individual (e.g., pyridine dilution series).

The article of manufacture can further comprise written or other format of instructions (e.g., C.D., video, cassette tapes, etc.) for use of the odorant composition for modifying an individual's balance time in a method according to the invention. The article of manufacture can further include one or more items for use in conducting the method of the invention, for example, a timing device such as a stopwatch, among other devices. In another embodiment, the article of manufacture can comprise packaging material and an odorant composition according to the invention contained within the packaging material, wherein the packaging material comprises a label that indicates that the odorant composition can be used for improving balance, e.g., increasing balance time in performing a balance activity. The article of manufacture can also include an odorant composition and instructions for testing olfactory threshold according methods known in the art. The parts of the article of manufacturing can be contained or separately packaged within a packaging material, such as a box, bag, pouch, and the like.

The invention will be further described by reference to the following detailed example. This example is not meant to limit the scope of the invention that has been set forth in the foregoing description. Variation within the concepts of the invention is apparent to those skilled in the art. The disclosures of the cited references throughout the application are incorporated by reference herein.

### EXAMPLE

### Methods.

Subjects underwent the Quick Smell Identification Test (Q-SIT) scratch-and-sniff test (Sensonics, Inc., Haddon Heights, N.J.). Eleven (11) normosmic (3/3 Q-SIT) subjects (3 males, 8 females, aged 20 to 58) underwent a test of stability by completing the Single Leg Balance Test using the Nintendo Wii Fit™ software in combination with the Nintendo Wii Balance Board according to the published protocol. The audio and visual feedback from the software was not made available to the subjects. The subjects were not blindfolded.

The subjects were instructed to use the non-dominant leg for the test. The dominant leg was defined as the leg the subject would use to kick a ball. In a consecutively alternating order, the subjects underwent testing while wearing blank surgical masks (as controls) or surgical masks impregnated with a cinnamon buns aroma (odorant) (i.e., a baked cinnamon buns aroma) (International Flavors & Fragrances Inc. (IFF)), in a counter balanced order.

### Results.

All eleven (11) subjects tested 3/3 on the Q-SIT Scratch and Sniff test. Ten (10) out of the eleven (11) subjects reported positive hedonics to the odor of the cinnamon buns aroma.

A comparison was made of the stability percentage using the cinnamon buns aroma (CBA) mask versus blank mask:

Means:

Under CBA: mean=67.1515, N=33 (11 individuals)

Under Blank: mean=60.151, N=33 (same 11 individuals)

The two means were compared using a mixed effects linear regression model with dependent variable stability percentage and a random intercept term to account for correlation amongst repeated measurements (N=66; 11 subjects with 6 measurements each). The effect of the factor “mask” was tested with an F-test and the result was significant (F=5.64; df=64, p=0.0205).

Stability percentage was significantly higher using the cinnamon buns aroma (67%) compared to the un-odorized blank mask (60%), with each person serving as their own control (p=0.02).

Order of presentation (odorant first or blank first) had no effect on the outcome (p=0.96). The model with “order” alone showed no effect of order (F(1.64)=0.00, p=0.9600). Adding a binary indicator for “order” to the model with “mask” was not significant (F(1.63)=0.00, p=0.9586).

The results also showed that gender had an effect on the outcome, with higher stability percentage in females, i.e., 66.7% females vs. 65.6% males (p=0.0009). In the model with gender alone predicting stability percentage, the significance of gender was F(1.64)=12.22, p=0.0009. In the model with mask alone predicting stability percentage, the significance of the mask was F(1.64)=5.64, p=0.0205. In the model with both gender and mask as independent variables:

Effect of gender, adjusted for mask: F(1.63)=13.32, p=0.0005

Effect of mask, adjusted for gender: F(1.63)=6.73, p=0.0118

Adjusted effects were slightly stronger than unadjusted effects, but very similar. The interaction between mask and gender was not significant (F(1.62)=1.48, p=0.2290).

The odorant-versus-blank effect was not different between men and women. The unadjusted effects of mask and gender were as follows:

| | 11 | 66 | | | | |
|---|---|---|---|---|---|---|
| | N¹ | N² | | | | |
| | (Number | (Number | | | | |
| | of. | of meas- | Stability Percentage | | | |
| Total: | subjects) | urements | Mean | SD | Minimum | Maximum |
| MASK | | | | | | |
| Odorant | 11 | 33 | 67.15 | 10.19 | 45 | 82 |
| Blank | 11 | 33 | 60.15 | 13.51 | 20 | 82 |
| GENDER: | | | | | | |
| Female | 8 | 48 | 66.67 | 11.00 | 36 | 82 |
| Male | 3 | 18 | 55.61 | 12.57 | 20 | 80 |

The results showed that the cinnamon buns odorant improved balance significantly in the test subjects compared to the blank.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents. The disclosures of the cited patents, applications, and other references throughout the application are incorporated by reference herein.

What is claimed is:

1. A method of improving balance of an individual, the method comprising administering to the individual for inhalation a composition comprising an odorant, wherein the inhalation of the odorant composition is effective to increase an amount of time the individual can maintain their balance while performing a balance activity compared to an amount of time they can maintain their balance while performing the same activity but without having inhaled the odorant composition.

2. The method of claim 1, wherein the composition comprises an effective amount of a cinnamon buns odorant to increase said time amount.

3. The method of claim 1, wherein the composition comprises an effective amount of a cinnamon odorant and a vanilla odorant to increase said time amount.

4. The method of claim 1, wherein the composition consists essentially of a cinnamon buns odorant in a carrier, and optional additives.

5. The method of claim 1, wherein the composition consists essentially of a mixture of a cinnamon odorant and a vanilla odorant in a carrier, and optional additives.

6. The method of claim 1, wherein the composition comprises a mixture of odorants in a carrier, said mixture of odorants comprising a suprathreshold and non-irritant concentration of a cinnamon buns odorant as a primary odorant, in combination with a less than suprathreshold concentration of one or more secondary odorants that complement and do not mask the cinnamon buns odorant, such that, when inhaled by an individual, the composition is effective to increase said time amount by a statistically significant amount.

7. The method of claim 1, wherein the composition comprises a mixture of odorants in a carrier, said mixture of odorants comprising a suprathreshold and non-irritant concentration of a mixture of a cinnamon odorant and a vanilla odorant as a primary odorant, in combination with a less than suprathreshold concentration of one or more secondary odorants that complement and do not mask the primary odorant, such that, when inhaled by an individual, the composition is effective to increase said time amount by a statistically significant amount.

8. The method of claim 1, wherein the composition comprises a concentration of the odorant greater than an average normal threshold concentration of the odorant.

9. The method of claim 1, wherein the composition comprises a suprathreshold but non-irritant concentration of the odorant.

10. The method of claim 1, wherein the composition comprises a concentration of the odorant of about 25-55 decismel units.

11. The method of claim 1, further comprising assessing said time amounts to determine effectiveness of administering the odorant composition to improve the individual's balance.

12. The method of claim 1, wherein the method is self-administered.

13. A method of improving balance of an individual, the method comprising:
inhaling a composition comprising an odorant; and
performing a balance activity;
wherein inhaling the odorant composition is effective to increase a time amount during which the individual maintains balance during said activity by a statistically significant amount compared to a time amount during which balance is maintained in performing said balance activity but without inhaling the odorant composition.

14. A method of improving balance of an individual, the method comprising:
administering to the individual for inhalation a composition comprising an odorant;
having the individual perform a balance activity with and without inhaling said composition; and
recording and comparing time amounts during which the individual maintains balance during said activity with and without inhaling said composition;
wherein the inhalation of the odorant composition is effective to increase the time amount by a statistically significant amount.

15. A method of improving balance of an individual, the method comprising the steps of:
(A) Having the individual perform a balance activity;
(B) Recording a first time amount during which the individual maintains balance during said activity;
(C) Administering to the individual for inhalation a composition comprising an odorant;
(D) Having the individual perform the balance activity;
(E) Recording a second time amount during which the individual maintains balance during said balance activity; and
(F) Scoring the difference between the first and second time amounts;
wherein the inhalation of the odorant composition is effective to increase the second time amount by a statistically significant amount compared to the first time amount.

16. The method of claim 15, wherein steps (C) and (D) are performed prior to steps (A) and (B).

17. An article of manufacture, comprising, packaged together:
a container of a composition comprising an odorant, and
instructions for administration of the composition to an individual by inhalation for improving the individual's balance.

18. The article of claim 17, wherein the composition comprises a cinnamon buns odorant as a primary odorant.

19. The article of claim 17, wherein the composition comprises a mixture of a cinnamon odorant and a vanilla odorant as a primary odorant.

20. A method of screening a test odorant composition for improving an individual's balance, the test odorant composition consisting essentially of the odorant or odorants to be tested, the method comprising the steps of:
having the individual perform a balance activity without inhalation of the test odorant composition and measuring the interval of time during which the individual maintains balance as a “control” time;
having the individual perform the same balance activity under the same conditions with inhalation of the test odorant composition and measuring the interval of time during which the individual maintains balance as a “test” time;
comparing the control time to the test time to determine the statistical significance of the test time;
assessing the composition as effective to improve the balance time of the individual based on said statistical significance of the test time; and
eliminating the odorant composition as ineffective to improve the individual's balance if the test time is not statistically significant (p<0.05).
